(19) United States
(12) Patent Application Publication
(10) Pub. No.: US 2002/0041908 A1
(43) Pub. Date: Apr. 11, 2002

(54) Iridacea extracts for stimulating the immune system

(76) Inventors: Lionel Breton, Versailles (FR); Audrey Gueniche, Velizy (FR)

Correspondence Address: Norman H. Stepno, Esquire, BURNS, DOANE, SWECKER & MATHIS, L.L.P., P.O. Box 1404, Alexandria, VA, 22313-1404, US

(21) Appl. No.: 09/906,753

(22) Filed: Jul. 18, 2001

(30) Foreign Application Priority Data
Jul. 18, 2000 (FR) 00/09404

(51) Int. Cl. A61K 35/78

(52) U.S. Cl. 424/725

(57) ABSTRACT

A regime or regimen for stimulating the immune defenses of individuals in need of such treatment, notably the immune defenses of the skin, comprises administering to such individuals, for such period of time as required to elicit the desired response, a thus effective amount of at least one extract of at least one plant of the Iridacea genera, for example of the genus Romulea, Iris, Crocus, Gladiolus, Sisyrinchium or Hermodactylus.

### CROSS-REFERENCE TO PRIORITY APPLICATION

This application claims priority under 35 U.S.C. §119 of FR-00/09404, filed Jul. 18, 2000, hereby expressly incorporated by reference.

### BACKGROUND OF THE INVENTION

1. Technical Field of the Invention

The present invention relates to the administration, whether orally or topically, of at least one extract of at least one plant of the Iridacea genera, or composition comprised thereof, for stimulating the immune defenses of an individual subject in need of such treatment.

The present invention also relates to a cosmetic regime/regimen for stimulating the immune function of the skin.

2. Description of the Prior Art

The immune system is known to comprise a set of specialized cells which are subjected to multiple mechanisms of control ensuring their renewal, their activation and their differentiation, and which are essential for a normal level of immunocompetence. The role of the immune system is to recognize self and non-self in order to eliminate pathogenic agents and spontaneous tumours. Any cellular depletion, any immune dysregulation or any functional deficiency is likely to promote the occurrence of pathological signs characterized by the disturbance of the mechanisms of recognition of self (native or natural) with respect to non-self (foreign), and a greater sensitivity with respect to microbial attacks and neoplastic processes.

The skin constitutes the largest organ of the human body and is recognized as an active element of the immune defense system. Three types of epidermal cell are involved in this system: keratinocytes, melanocytes and Langerhans cells. These cells, which are only found in the epidermis, play a vital role in the immune response of the skin.

Healthy skin is capable of defending itself against external attacks using the mechanisms available thereto. However, it is subjected to constant attack or challenge from the environment (infections and pollution in particular), from chemical products and from radiation. In particular, the Langerhans cells are the preferred target of ultraviolet radiation.

These attacks initiate a suppresser effect on the immune defenses, promoting a decrease in the resistance to pathogenic agents and, in particular, an increase in the incidence of certain cancers.

In order to assist the skin in accomplishing its immune function, products or bioaffecting agents which regulate the immune system of the skin are of great value.

In light of the above, it is now known to this art that the immune system, and more particularly that of the skin, becomes weaker during chronobiological aging or photoinduced aging.

An immunoregulatory effect applied to this immune system may, therefore, re-establish immune functions, and more particularly those of the epidermis, which contributes towards restoring the natural defenses of the skin and thus preventing certain pathogenic disorders and the appearance of cancers.

### SUMMARY OF THE INVENTION

Accordingly, a major object of the present invention is the provision of a novel stimulatory immunoregulator, more particularly a regulator which stimulates the immune system of the skin and which is compatible therewith.

Briefly, it has now unexpectedly and surprisingly been determined that administering an effective amount of at least one extract of at least one plant of the Iridacea genera stimulates the immune defenses, upon attack or challenge thereto, particularly the immune defenses of the skin.

Administration of the extract is particularly effective in stimulating the immune defenses of the skin when they are weakened by exposure to ultraviolet radiation, inducing, over the long term, photoinduced skin aging.

Thus, the present invention features administration of at least one extract of at least one plant of the Iridacea genera, or composition comprised thereof, to an individual subject in need of such treatment for stimulating the immune defenses of such individual, particularly the immune defenses of the skin.

The present invention also features administration of at least one extract of at least one plant of the Iridacea genera, or composition comprised thereof, for stimulating immune defenses weakened by the processes or cascade of photoinduced aging.

### DETAILED DESCRIPTION OF BEST MODE AND SPECIFIC/PREFERRED EMBODIMENTS OF THE INVENTION

More particularly according to the present invention, the Iridacea family includes approximately 750 species. The plants of the Iridacea family are especially useful for their aromatic and ornamental properties.

Among the Iridacea genera which are well suited according to the invention, exemplary are the Romulea, Crocus, Iris, Gladiolus, Sisyrinchium and Hermodactylus genera.

A preferred plant material is that which originates from the Iris germanica, Iris florentina, Iris pallida, Crocus versicolor, Romulea bulbucodium and Gladiolus communis plant species.

More particularly according to this invention, plant material derived from the Iris genus, and preferably plant material derived from the Iris pallida, Iris germanica and Iris florentina species, is employed.

The extract of at least one Iridacea can be any extract prepared from any plant material derived from the Iridacea family.

The plant material may originate from whole plants cultivated in vivo or derived from culturing in vitro. The selection pressure imposed by the physicochemical conditions during the growth of the plant cells in vitro makes it possible to obtain a standardized plant material which is available throughout the year, unlike the plants cultivated in vivo.

By the expression “culturing in vitro” are intended all of the techniques known to this art which make it possible to artificially produce a plant or a part of a plant.

Preferably, an extract obtained from plant material cultured in vitro is employed.

Useful plant material according to the invention can, for example, be derived from a whole plant or from a part of a plant, such as the leaves, the stems, the flowers, the petals, the roots, the fruits, the seeds or the anthers, or even from dedifferentiated plant cells.

By the expression “dedifferentiated plant cells” is intended any plant cell which exhibits none of the characteristics of a particular specialization and which is capable of living and survival by itself and in a situation where it is not dependent on other cells. These dedifferentiated plant cells are possibly capable, under the effect of an induction, of any differentiation in accordance with their genome.

The dedifferentiated plant cells can be obtained from plant material derived from a whole plant or from a part of a plant, such as the leaves, the stems, the flowers, the petals, the roots, the fruits, the seeds or the anthers, whether the plant or the part of a plant has been obtained by cultivation in vivo or cultivation in vitro.

Preferably, the dedifferentiated plant cells are obtained from leaves or from rhizomes.

Preferably, according to the invention, dedifferentiated plant cells derived from cultivation in vitro are employed.

Very preferably, dedifferentiated plant cells derived from cultivation in vitro, obtained from leaves or from rhizomes of a plant of the Iris pallida, Iris germanica and Iris florentina species are employed.

The dedifferentiated plant cells which are suitable according to the invention can be obtained via any known method of the prior art. In this regard, reference is made to the techniques described by E. F. George and P. D. Sherrington in “Plant Propagation by tissue culture, handbook and directory of commercial laboratories” (Exegetics Ltd 1984).

The culture media which are suitable according to the invention are those generally known to those skilled in this art. Compare, for example, the media of Gamborg, Murashige and Skoog, Heller, White, etc. Complete descriptions of these media are available in “Plant Culture Media: formulations and uses” by E. F. George, D. J. M. Puttock and H. J. George (Exegetics Ltd 1987, volumes 1 and 2).

Preferably, the cultured dedifferentiated plant cells are prepared on Murashige and Skoog medium.

Any extraction technique known to this art can be used to prepare the Iridacea extract according to the invention. Exemplary are, in particular, alcoholic extractions, in particular ethanolic or aqueous/alcoholic extractions.

An Iridacea extract can also be used that is prepared using the method described in French patent application No. 95-02379, assigned to the assignee hereof. Thus, in a first step, the plant material is ground in an aqueous solution while cold, in a second step, the particles in suspension are removed from the aqueous solution derived from the first step and, in a third step, the aqueous solution derived from the second step is sterilized.

This aqueous solution corresponds to the extract.

On the other hand, the first step may be advantageously replaced with a simple procedure for freezing the plant tissues (for example at −20° C.), followed by an aqueous extraction repeating the second and third steps described above. The product of this aqueous extraction can also be lyophilized so as to obtain a dry extract (on average, depending on the nature of the initial source, 5 to 70 g of solids are obtained per liter of aqueous extract).

One example of preparation of an Iridacea extract according to the invention is given in the examples to follow.

The amount of extract administered according to the invention depends, of course, on the desired effect and may therefore vary over wide limits.

To provide an order of magnitude, a dry extract as described above can be administered in an amount representing from 0.0001% to 20% of the total weight of the composition, and preferably in an amount representing from 0.001% to 10% of the total weight of the composition.

The compositions of the invention are suitable for cosmetic or dermatological (therapeutic) applications.

Preferably, the subject compositions are compositions for cosmetic applications.

The compositions according to the invention can be provided in all of the pharmaceutical forms normally employed for topical or oral administration.

According to the invention, cosmetic compositions for topical application are the preferred.

For topical application, the compositions of the invention comprise a medium (vehicle, diluent or carrier) which is compatible with the skin. These compositions can, in particular, be in the form of aqueous, alcoholic or aqueous/alcoholic solutions, of gels, of water-in-oil or oil-in-water emulsions having the appearance of a cream or of a gel or ointment, of microemulsions or of aerosols, or in the form of vesicular dispersions containing ionic and/or nonionic lipids. These pharmaceutical forms are prepared via the conventional methods of the fields under consideration.

These compositions for topical application may, in particular, constitute a cosmetic or pharmaceutical protective, treatment or care composition for the face, for the neck, for the hands or for the body (for example day creams, night creams, sunscreen creams or oils, or body milks), a makeup composition (for example a foundation), or an artificial tanning composition.

The amounts of the various constituents of the compositions according to the invention are those conventionally included in the fields under consideration and are suitable for their pharmaceutical form.

When the composition of the invention is an emulsion, the proportion of fatty substance it contains may range from 5% to 80% by weight, and preferably from 5% to 50% by weight, with respect to the total weight of the composition. The fatty substances and emulsifiers included in the composition in emulsion form are selected from among those conventionally formulated in the cosmetics or pharmaceutical field.

Exemplary such fatty substances include the mineral oils (petroleum jelly), plant oils (liquid fraction of karite butter) and hydrogenated derivatives thereof, animal oils, synthetic oils (perhydrosqualene), silicone oils (polydimethylsiloxane) and fluoro oils. Other fatty substances which are exemplary are the fatty alcohols (cetyl alcohol or stearyl alcohol), fatty acids (stearic acid) and waxes.

The emulsifiers are advantageously present in the composition in a proportion ranging from 0.3% to 30% by weight, and preferably from 0.5% to 30% by weight, with respect to the total weight of the composition.

In known fashion, the compositions of the invention may also contain additives and adjuvants which are conventional in the corresponding fields, such as hydrophilic or lipophilic gelling agents, preservatives, antioxidants, solvents, fragrances, fillers, screening agents and dyestuffs and colorants. Moreover, these compositions may contain hydrophilic or lipophilic active agents. The amounts of these various additives, adjuvants or active agents are those conventional in the cosmetics or pharmaceutical field, and range, for example, from 0.01% to 20% of the total weight of the composition. Depending on their nature, these additives, adjuvants or these active agents may be introduced into the fatty phase, into the aqueous phase and/or into lipid vesicles.

Among the active agents which the compositions of the invention may contain, particularly exemplary such active agents are those for treating wrinkles or fine lines, and in particular keratolytic active agents. By the term “keratolytic” is intended an active agent which has desquamating, exfolient or scrubbing properties, or an active agent capable of decreasing the cohesion of the horny layer.

Exemplary active agents for treating wrinkles and fine lines include, in particular, hydroxy acids and retinoids.

The hydroxy acids may be, for example, α-hydroxy acids or β-hydroxy acids, which may be linear, branched or cyclic, and saturated or unsaturated. The hydrogen atoms of the carbon-based chain may also be substituted with halogens or halogenated, alkyl, acyl, acyloxy, alkoxycarbonyl or alkoxy radicals having from 2 to 18 carbon atoms.

Particularly exemplary hydroxy acids include glycolic acid, lactic acid, malic acid, tartric acid, citric acid, 2-hydroxyalcanoic acid, mandelic acid, salicylic acid and the alkyl derivatives thereof, for example 5-n-octanoylsalicylic acid, 5-n-dodecanoylsalicylic acid, 5-n-decanoylsalicylic acid, 5-n-octylsalicylic acid, 5-n-heptyloxysalicylic acid or 4-n-heptyloxysalicylic acid, and 2-hydroxy-3-methylbenzoic acid or alkoxylated derivatives thereof, for example 2-hydroxy-3-methoxybenzoic acid.

An particulary exemplary retinoids include retinoic acid (all-trans or 13-cis) and derivatives thereof, retinol (vitamin A) and esters thereof, such as retinol palmitate, retinol acetate and retinol propionate, and salts thereof.

Among the active agents which the compositions according to the invention may contain, particularly desirable are active agents which will contribute to protecting the immune system.

Exemplary active agents contributing to protecting the immune system include those that are free-radical scavengers, such as, for example, provitamin A carotenoids, e.g., beta-carotene, non-provitamin A carotenoids, e.g., lycopene, vitamin E and derivatives thereof, vitamin C and derivatives thereof, and inorganic and organic sunscreens.

Also among the active agents which the compositions according to the invention may contain are the sunscreens and active agents which protect the extracellular matrix proteins from UV-induced damage, such as inhibitors of UV-induced proteases, in particular metalloproteinase 1 inhibitors; such enzymatic inhibition may be on the enzymatic activity but also on the synthesis of the enzyme itself. Particularly exemplary anti-AP1s are described in U.S. Pat. No. 5,837,224 to J. Voorhees et al.

These active agents are advantageously formulated at concentrations ranging from 0.0001% to 5% by weight with respect to the total weight of the composition.

When the compositions of the invention are for oral administration, they may be in any pharmaceutical form which is conventional in this field, such as tablets, gelatin capsules, drinkable products, in particular products reconstituted extemporaneously, granules or powders, formulated into the usual excipients for such application (vehicle, diluent or carrier therefor).

The present invention thus a cosmetic regime/regimen for stimulating the immune defenses of the skin, comprises applying topically, or ingesting, for such period of time as required to elicit the desired response, a cosmetic composition which comprises at least one extract of at least one Iridacea as described above.

In order to further illustrate the present invention and the advantages thereof, the following specific examples are given, it being understood that same are intended only as illustrative and in nowise limitative.

In said examples to follow, all parts and percentages are given by weight, unless otherwise indicated.

### EXAMPLE 1

Preparation of an extract of Iris pallida:

Dedifferentiated Iris pallida cells cultured in vitro under axenic conditions were recovered, after culturing in Erlenmeyer flasks or in fermenters, by filtration through a 50 μm sieve. 27.5 ml of demineralized water were added to 55 g of fresh material thus obtained. The mixture was ground in a Turax blender at 24,000 rpm for 1 minute at 4° C. (ice bath). The ground material was centrifuged 15 minutes at 10,000 G at 4° C.

The supernatant was filtered at 0.22 μm (sterilizing filtration).

The extract thus prepared was stored at 4° C. It contained approximately 15 g of solids per liter, and it could be lyophilized to obtain a dry extract.

If the plant material was from the whole plant, the fresh material to be treated was corrected as a function of the dry weight so as to operate under the same extraction conditions as for the in vitro material. The various parts of the plant were removed as a function of the relative weight of each part of the plant. The cold treatment made it possible to freeze the enzymatic activities and the sterilizing filtration avoided degradation of the active agents by the microorganisms of the environment. Finally, the water vehicle was compatible with ex vivo receptors and facilitated cosmetic and pharmaceutical formulations. The extract could also be used in the form of a dry extract.

### EXAMPLE 2

Activity of an extract of Iris pallida (dry extract obtained by lyophilization of the aqueous extract described in Example 1) on the protection of the immune system of the skin, depressed after UV-radiation:

The extract of Iris pallida was applied to Skh/hr1 hairless mice after irradiation using a sun simulator (Xenon 1000 W (Oriel) equipped with filters (Oriel 81017+81019)) at a dose known to cause inhibition of contact hypersensitivity (CHS). The irradiation, measured with an ARCC 1600/OSRAM radiometer, was 2.0 mW/cm² for UVB and 9.4 mW/cm² for UVA. Skin biopsies were then taken 14 days after irradiation.

The products applied were formulated in a simple support:

(a) Control vehicle: AMPS-604518 gel, marketed by CLARIANT (FRANCE) under the trademark HOSTACERIN AMPS®;

(b) Iris pallida at 5% (dry extract) in an AMPS-604518 gel.

The results obtained evidenced that, in the system employed (Skh/hr1 hairless murine strain, sun simulator (acute irradiation at a dose of 2.5 MED), systemic CHS to DNFB), the application of a dry extract of Iris pallida formulated at 5% in an AMPS gel significantly attenuated the immunosuppressive effects engendered by UV radiation, measured by a CHS reaction to dinitro-fluorobenzene (DNFB).

Indeed, the products, without having any stimulatory activity on the CHS reaction, reduced the photoimmunosuppression.

In parallel to this clinical examination, a histological/biochemical assessment (number, morphology) of the Langerhans cells was carried out.

In the controls which were not protected with Iris pallida, approximately 50% of the number of Langerhans cells disappeared after UV irradiation, with, for those which remained, significant adverse modifications at the cell surface and in terms of morphology.

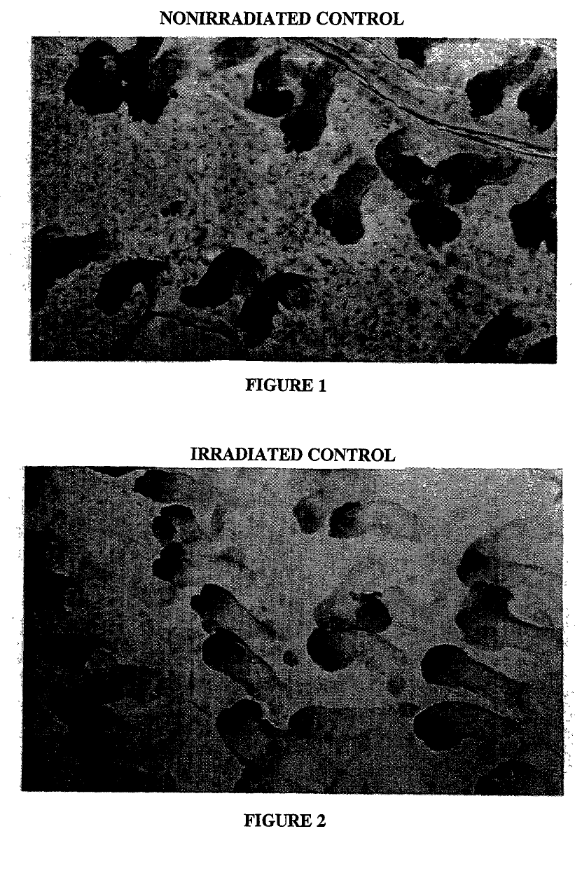

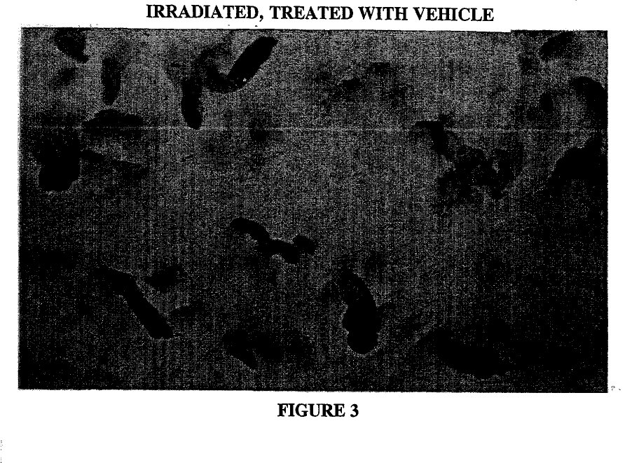

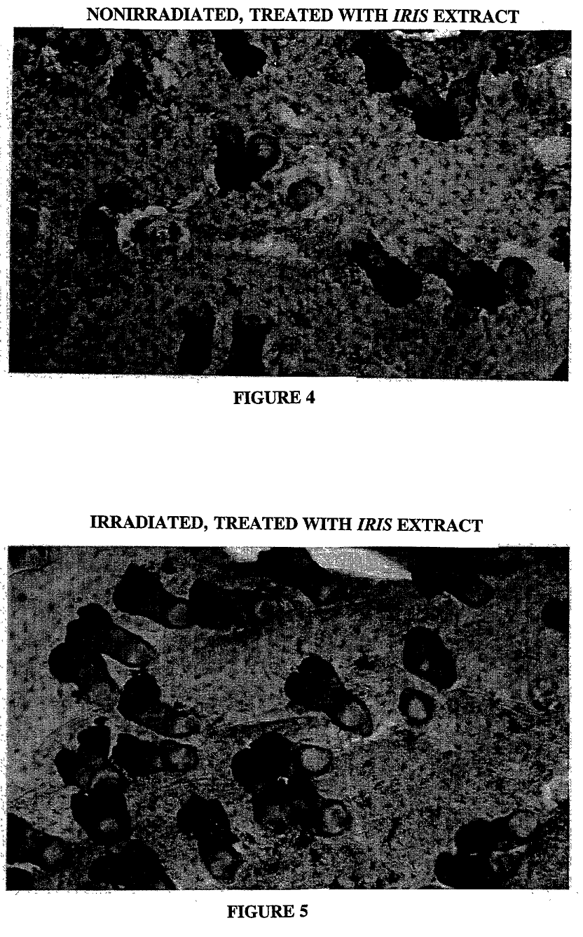

The treatment with the extract of Iris pallida protected the skin against the irradiation-induced adverse modification of the Langerhans cells, since the number and morphological appearance of these cells was identical to the control (cf. attached photomicrographs of FIGS. 1-5 and Table below).

Quantification of Langerhans cells during the treatment, given as number of Langerhans cells per mm²:

TABLE

| Non-irradiated | Irradiated | Non-irradiated | Irradiated |
|---|---|---|---|
| controls | controls | treated | treated |
| 1406 ± 82 | 720 ± 36 | 1334 ± 97 | 1515 ± 112 |

In the irradiated controls, approximately 50% of the Langerhans cells disappeared. This disappearance was inhibited by applying a gel containing 5% of a dry extract of Iris pallida. Moreover, application of the 5% Iris pallida gel only exerted an influence on the immune system of the skin when the latter was adversely modified by UV radiation.

### EXAMPLES 3-7

The following Examples 3-7 are of specific compositions according to the present invention, formulated as indicated. These compositions were formulated by simply intimately admixing the individual constituents thereof.

### EXAMPLE 3

Composition 1—Milk for the face:

| | |
|---|---|
| Liquid petroleum jelly | 7.0% |
| Extract of Example 1 (lyophilized extract) | 5.0% |
| Glyceryl monostearate, polyethylene | 3.0% |
| glycol stearate (100 EO) | |
| Carboxyvinyl polymer | 0.4% |
| Stearyl alcohol | 0.7% |
| Soya bean proteins | 3.0% |
| NaOH | 0.4% |
| Preservative | q.s. |
| Water | q.s. for 100.0% |

This composition was formulated as a milk for the face; it had good cosmetic properties and was soft and comfortable to use.

The pH of the composition was approximately 5.5.

### EXAMPLE 4

Composition 2—Lotion:

| | |
|---|---|
| Extract of Example 1 (lyophilized extract) | 2.0% |
| 2-ethylhexyl palmitate | 10.0% |
| Cyclopentadimethylsiloxane | 20.0% |
| Butylene glycol | 5.0% |
| Preservative | q.s. |
| Water | q.s. for 100.0% |

This lotion, which did not contain any surfactant, promoted desquamation of the skin.

### EXAMPLE 5

Composition 3—Milk:

| | |
|---|---|
| Octyl palmitate | 35.0% |
| Glycerol | 2.0% |
| Extract of Example 1 | 3.0% |
| (lyophilized extract) | |
| C₁₀-C₃₀ acrylates/alkyl acrylates | 0.1% |
| crosslinked polymer | |
| Triethanolamine | 0.1% |
| Wheat amino acids | 1.0% |
| Preservative | q.s. |
| Water | q.s. for 100.0% |

The milk obtained, which did not contain any surfactant, had good cosmetic properties.

### EXAMPLE 6

Composition 4—Gel for the face:

| | |
|---|---|
| Glycerol | 10.0% |
| Extract of Example 1 | 5.0% |
| (lyophilized extract) | |
| Disodium cocoamphodiacetate | 1.0% |
| Preservative | q.s. |
| Water | q.s. for 100.0% |

The gel obtained had good cosmetic properties.

### EXAMPLE 7

Composition 5—Gel for cleansing with water:

| | |
|---|---|
| Butylene glycol | 7.0% |
| Sodium lauroyl sarcosinate | 4.0% |
| Extract of Example 1 | 1.5% |
| (lyophilized extract) | |
| Triethanolamine | 0.8% |
| Carbomer | 0.5% |
| Preservative | q.s. |
| Water | q.s. for 100.0% |

The gel obtained had good cosmetic properties.

While the invention has been described in terms of various specific and preferred embodiments, the skilled artisan will appreciate that various modifications, substitutions, omissions, and changes may be made without departing from the spirit thereof. Accordingly, it is intended that the scope of the present invention be limited solely by the scope of the following claims, including equivalents thereof.

What is claimed is:

1. A regime/regimen for stimulating the immune defenses of an individual subject in need of such treatment, comprising administering to such individual, for such period of time as required to elicit the desired response, a thus effective amount of at least one extract of at least one plant of the Iridacea genera.

2. A regime/regimen for stimulating the immune defenses of the skin of an individual subject in need of such treatment, comprising administering to such individual, for such period of time as required to elicit the desired response, a thus effective amount of at least one extract of at least one plant of the Iridacea genera.

3. A regime/regimen for stimulating the photoinduced aging-weakened immune defenses of an individual subject in need of such treatment, comprising administering to such individual, for such period of time as required to elicit the desired response, a thus effective amount of at least one extract of at least one plant of the Iridacea genera.

4. The regime/regimen as defined by any of claims 1 to 3, said at least one extract having been obtained from at least one plant of the Iridacea genera cultivated in vitro.

5. The regime/regimen as defined by any of claims 1 to 3, said at least one extract having been obtained from at least one plant of the Iridacea genera cultivated in vivo.

6. The regime/regimen as defined by any of claims 1 to 3, said at least one extract of at least one plant of the Iridacea genera having been obtained from the dedifferentiated cells thereof.

7. The regime/regimen as defined by any of claims 1 to 3, said at least one plant of the Iridacea genera being of the genus Romulea, Crocus, Iris, Gladiolus, Sisyrinchium or Hermodactylus.

8. The regime/regimen as defined by claim 7, said at least one plant of the Iridacea genera being of the Iris genus.

9. The regime/regimen as defined by claim 8, said at least one plant of the Iris genus being Iris pallida, Iris florentina or Iris germanica.

10. The regime/regimen as defined by claim 1, comprising orally administering said at least one extract of at least one plant of the Iridacea genera to such individual subject in need of such treatment.

11. The regime/regimen as defined by claim 2, comprising topically applying said at least one extract of at least one plant of the Iridacea genera onto the skin of such individual subject in need of such treatment.

12. A topically applicable cosmetic/therapeutic composition for stimulating the immune defenses of the skin, comprising from 0.0001% to 20% by weight of at least one extract of at least one plant of the Iridacea genera, formulated into a topically applicable, cosmetically/therapeutically acceptable vehicle, diluent or carrier therefor.

13. The topically applicable cosmetic/therapeutic composition as defined by claim 12, comprising from 0.001% to 10% by weight of said at least one extract of at least one plant of the Iridacea genera.

14. The topically applicable cosmetic/therapeutic composition as defined by claim 12, formulated as an emulsion, cream, gel, lotion, milk, sunscreen, ointment or aerosol.

15. An orally ingestable cosmetic/therapeutic composition for stimulating the immune defenses, comprising from 0.0001% to 20% by weight of at least one extract of at least one plant of the Iridacea genera, formulated into an orally ingestable, cosmetically/therapeutically acceptable vehicle, diluent or carrier therefor.

16. The orally ingestable cosmetic/therapeutic composition as defined by claim 15, comprising from 0.001% to 10% by weight of said at least one extract of at least one plant of the Iridacea genera.

17. The orally ingestable cosmetic/therapeutic composition as defined by claim 15, formulated as tablets, granules, capsules, a powder, or a drinkable product.

18. A cosmetic/therapeutic composition as defined by claims 12 or 15, further comprising an effective amount of at least one keratolytic active agent.

19. A cosmetic/therapeutic composition as defined by claim 18, said at least one keratolytic active agent comprising a hydroxy acid, a retinoid, or combination thereof.

20. A cosmetic/therapeutic composition as defined by claims 12 or 15, further comprising a free-radical scavenger, a provitamin A carotenoid, a non-provitamin A carotenoid, vitamin E or derivative thereof, vitamin C or derivative thereof, a sunscreen, an active agent which protects extracellular matrix proteins from UV-induced photodamage, an inhibitor of UV-induced proteases or combination thereof.
